(19) United States
(12) Patent Application Publication
(10) Pub. No.: US 2007/0196418 A1
(43) Pub. Date: Aug. 23, 2007

(54) Drug delivery methods and devices

(76) Inventors: Michael Lewis, Andover, MA (US); Bruce A. Littlefield, Andover, MA (US); J. Paul Carter, North Andover, MA (US)

Correspondence Address: CLARK & ELBING LLP, 101 FEDERAL STREET, BOSTON, MA, 02110, US

(21) Appl. No.: 10/566,331

(22) PCT Filed: Jul. 29, 2004
(86) PCT No.: PCT/US04/24550

§ 371 Date: Feb. 14, 2007

Related U.S. Application Data
(60) Provisional application No. 60/490,757, filed on Jul. 29, 2003.

(51) Int. Cl. A61F 2/82; A01N 43/16

(52) U.S. Cl. 424/423; 514/183; 514/459

(57) ABSTRACT

The invention provides devices and methods for delivering therapeutic agents to patients.

### FIELD OF THE INVENTION

This invention relates to methods and devices for use in administering therapeutic agents.

### BACKGROUND OF THE INVENTION

Coronary artery disease is a leading cause of death and debilitation in the western world. It is estimated that 1,500,000 new or recurrent heart attacks due to coronary artery disease occur each year in the United States alone, where this disease affects close to 60 million people. Further, up to 50% of people in the United States may be at risk for coronary artery disease by having high cholesterol levels. Coronary artery disease is caused by atherosclerosis, which is a chronic condition characterized by the abnormal thickening and hardening of arterial walls. Atherosclerosis is characterized by the accumulation of substances such as lipids, cholesterol, calcium, fibrin, and cellular debris within the arterial wall intima, where together these substances form lesions referred to as plaque.

In addition to being a cause of heart disease, such as coronary artery disease, atherosclerosis is a systemic condition that can affect all major arteries, including those in the brain, the kidneys, and the extremities. Thus, in addition to coronary artery disease, atherosclerosis can cause diseases and conditions such as carotid artery disease, angina pectoris, pulmonary artery stenosis, cerebral vascular disease, thrombotic stroke, transient ischemia, diabetic vascular complications, gangrene of the extremities, and other forms of peripheral vascular disease. Further, atherosclerosis can also occur in coronary artery bypass grafts (CABG). Risk factors for atherosclerosis include elevated levels of cholesterol and triglycerides in the blood, high blood pressure, and cigarette smoking.

A very widely used approach to treating atherosclerosis is percutaneous transluminal angioplasty (PTA) or, in the case of treating atherosclerosis of the coronary arteries, percutaneous transluminal coronary angioplasty (PTCA). These approaches involve the insertion of a balloon catheter into a blocked artery and inflation of the balloon to compress plaque deposits within the inner wall of the artery, thereby improving blood flow. Although revolutionary in the field of cardiac and vascular medicine, angioplasty is not without risks. In particular, angioplasty is associated with the possibility of collapse of treated blood vessels. Further, within six months of the procedure, the treated arteries of 30-50% of angioplasty patients undergo the process of restenosis, which is the reoccurrence of stenosis (i.e., narrowing) in a blood vessel after angioplasty treatment. The narrowing that occurs in the process of restenosis is due to the hyperproliferation of vascular smooth muscle cells that is triggered by perturbation of the blood vessel by the balloon catheter.

An approach to overcoming the problem of restenosis in angioplasty involves the use of stents. A stent is cylindrical device, such as a wire mesh tube, that can be used to prop open an angioplasty-treated artery, to reduce the likelihood of restenosis. In particular, stents can be mounted on angioplasty balloons and delivered through catheters to diseased areas in arteries. Once placed at the site of a lesion within a blood vessel, the balloon is inflated, which also results in expansion of the mesh-like structure of the stent. The stent is thus pressed into place against the artery wall, where it acts as a vascular support. When the balloon is later deflated and withdrawn, the stent remains in place, serving as a permanent “scaffolding” for the newly unblocked artery. The natural lining of the artery then grows over the surface of the stent within a few weeks.

Intraarterial stents have had a major impact on the treatment of restenosis, such that they now are used in up to 80% of PTCA procedures. However, as with PTCA alone, the use of stents is associated with a significant incidence (15-25%) of restenosis (so-called in-stent stenosis), in which the stent itself induces proliferation of vascular smooth muscle cells that leads to re-occlusion of the affected vessel.

### SUMMARY OF THE INVENTION

The invention provides methods for inhibiting the growth of cells (e.g., vascular smooth muscle cells) in blood vessels (e.g., coronary arteries, vein grafts, and peripheral arteries), involving contacting the cells with halichondrin analogs. The blood vessels can be present in patients or can be treated ex vivo. In one example, the cells of the blood vessel are contacted with a halichondrin analog by the use of a stent that is inserted into the blood vessel. The halichondrin analog can be coated onto the stent, for example, it can be present in a polymeric matrix on the surface of the stent, and the matrix can facilitate release of the halichondrin analog from the matrix over time after insertion of the stent into the blood vessel. Optionally, the stent can further include one or more additional therapeutic agents, such as, for example, taxol, rapamycin, or heparin. The invention also includes the use of halichondrin analogs (and stents) for use in the methods described herein, as well as the use of the analogs (and stents) in the preparation of medicaments for use in these methods.

The halichondrin analogs used in the methods of the invention can be within the formula:

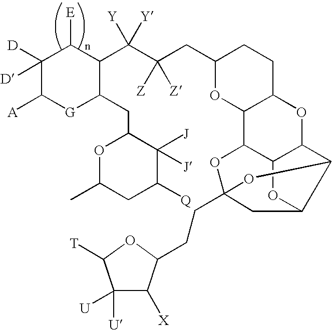

wherein A is a C₁₋₆ saturated or C₂₋₆ unsaturated hydrocarbon skeleton, the skeleton being unsubstituted or having between 1 and 10 substituents, inclusive, independently selected from cyano, halo, azido, oxo, and Q₁;

each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁, and O(CO)NR₂R₁;

each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl, C₆₋₁₀ hydroxyaryl, C₁₋₃ alkoxy-C₆ aryl, C₆₋₁₀ aryl-C₁₋₆ alkyl, C₁₋₆ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆ aryl)-C₁₋₃ alkyl, C₂₋₉ heterocyclic radical, C₂₋₉ heterocyclic radical-C₁₋₆ alkyl, C₂₋₉ hydroxyheterocyclic radical, C₂₋₉ heterocyclic radical-C₁₋₃ alkylhydroxy, C₂₋₉ heteroaryl, and C₂₋₉ heteroaryl-C₁₋₆ alkyl;

each of D and D′ is independently selected from R₃ and OR₃, wherein R₃ is H, C₁₋₃ alkyl, or C₁₋₃ haloalkyl;

n is 0 or 1;

E is R₅ or OR₅;

G is O, S, CH₂, or NR₆;

each of J and J′ is independently H, C₁₋₆ alkoxy, or C₁₋₆ alkyl; or J and J′ taken together are ═CH₂ or —O-(straight or branched C₁₋₅ alkylene)-O—;

Q is C₁₋₃ alkyl;

T is ethylene or ethenylene, optionally substituted with (CO)OR₇, where R₇ is H or C₁₋₆ alkyl;

each of U and U′ is independently H, C₁₋₆ alkoxy, or C₁₋₆ alkyl; or U and U′ taken together are ═CH₂ or —O-(straight or branched C₁₋₅ alkylene)-O—;

X is H or C₁₋₆ alkoxy;

each of Y and Y′ is independently H or C₁₋₆ alkoxy; or Y and Y′ taken together are ═O, ═CH₂, or —O-(straight or branched C₁₋₅ alkylene)-O—; and

each of Z and Z′ is independently H or C₁₋₆ alkoxy; or Z and Z′ taken together are ═O, ═CH₂, or —O-(straight or branched C₁₋₅ alkylene)-O—;

or a pharmaceutically acceptable salt thereof.

As a specific example, the halichondrin analog can have the structure:

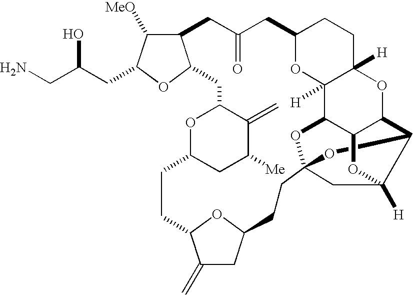

The invention also provides stents that include halichondrin analogs coated on their surfaces. For example, the analogs can be present in a polymeric matrix on the surface of the stent, and the matrix can facilitate the release of the halichondrin analog from the matrix over time after insertion of the stent into a blood vessel. Optionally, the stents of the invention can further include one or more additional therapeutic agents, such as, for example, taxol, rapamycin, or heparin. The halichondrin analogs included on the stents of the invention fall within the formula set forth above, and can thus be of the structure set forth above.

The invention provides several advantages. The methods and devices of the invention provide approaches to minimizing the possibility of restenosis following angioplasty treatment. Further, administration of halichondrin analogs using stents enables the use of substantially less drug than would be required for systemic administration, in an effort to achieve a similar effect.

Other features and advantages of the invention are present in the following detailed description and the claims.

### DETAILED DESCRIPTION OF THE INVENTION

The invention provides methods and devices for use in preventing or decreasing the growth of cells. The methods involve the administration of halichondrin analogs to cells to decrease the rate of or, preferably, prevent their growth. The methods and devices of the invention can be used in treating several different diseases associated with undesired hyperproliferation of cells, as is discussed further below.

In one example of the methods of the invention, halichondrin analogs are used to prevent the growth of vascular smooth muscle cells, as well as other cells that may play a role in restenosis, such as platelets and white blood cells, in blood vessels. As is discussed above, the growth of such cells can be triggered by angioplasty treatment, and can lead to blockage of the treated blood vessels. Blood vessels that are treated according to the invention can be those that are present in the heart, such as coronary arteries or bypass grafts (e.g., saphenous vein grafts). In the case of coronary artery bypass grafts, these blood vessels can be treated with a halichondrin analog before or after implantation into the heart. If treated before implantation, the drug can be applied to the interior surface of the graft or the graft can be bathed in a solution of the drug. If treated after implantation, the stent and/or balloon-based approaches described elsewhere herein can be employed.

In addition to being used to treat blood vessels of the heart, the methods and devices of the invention can be used in the peripheral vasculature, e.g., to treat peripheral vascular disease (of, e.g., the carotid or femoral arteries, or in diabetic patients), to prevent aneurysm rupture, or to treat renal artery stenosis. Indeed, the methods and devices of the invention can be used in the prevention and treatment of any condition that is characterized by undesired cell proliferation, such as that in a cylindrical tissue, e.g., a blood vessel or a duct.

Halichondrin analogs can be administered, according to the methods of the invention, using standard methods, such as by the use of stents and/or balloon catheters. As is discussed above, stents are mesh tubes or coils that are used to hold open the lumen of a cylindrical tissue, such as a blood vessel (e.g., an angioplasty-treated blood vessel), to reduce the likelihood of collapse or restenosis. For use in the present invention, stents are coated with one or more halichondrin analogs, either by dipping the stent in a solution containing the drug or, preferably, by the application of a polymer (e.g., a polymer that facilitates release of the drug over time) containing the drug to the stent. Methods for applying such coatings to the surfaces of stents for use in drug delivery are well known in the art (see, e.g., WO 98/57671, U.S. Ser. Nos. 08/526,273, 08/424,884, and 08/663,518, and U.S. Pat. Nos. 5,092,877, 4,916,193, 4,994,071, 5,304,121, 5,464,650, 5,282,823, 5,163,958, 5,342,348, 5,383,928, 5,575,818, and 6,358,556, the teachings of each of which are incorporated herein by reference), and can readily be adapted for use with halichondrin analogs by those of skill in the art. Alternatively, the drug can be contained within one or more reservoirs within the structure of a stent (see, e.g., U.S. Pat. No. 6,273,913). Further, the stent itself can be made of a material that serves as a matrix for the drug (see, e.g., U.S. Pat. No. 5,163,952).

Appropriate amounts of drug to be included in or on the stents of the invention can be determined by those of skill in the art. For example, the stents can be used to administer 0.1-100 μg, e.g., 1-50 μg or 5-15 μg of the drug.

Stents that are used in the invention can be delivered to a desired site (e.g., the site of a vascular lesion or an aneurysm) using any of a number of methods that are well known in the art, which include the use of balloon catheters, guide wires, and other delivery devices. As a specific example, a stent can be mounted on an angioplasty balloon and delivered by a catheter to a desired site. Once placed at such a site, the balloon can be inflated, which can also result in expansion of the mesh-like structure of the stent. The stent is thus pressed into place against the blood vessel wall, where it acts as a vascular support. As another example, a stent that is self-expanding (i.e., a stent that does not require expansion by use of a balloon) can be used. Approaches for delivery of such stents to desired locations are well known in the art (see, e.g., U.S. Pat. No. 6,019,778).

In addition to being administered using stents or stent/balloon catheter combinations, halichondrin analogs can be administered using balloon catheters alone, without stents. For example, the analogs can be released from a catheter between two occlusion balloons of the catheter or, preferably, can be pressed into blood vessel walls by being present as a coating on the surface of a balloon that is inflated within a blood vessel. The latter possibility can take place at the same time as an original angioplasty procedure or can take place later, for example, if symptoms consistent with the possibility of restenosis are observed. As a specific example of balloon-based approaches, applicants refer to U.S. Pat. No. 6,146,358, which describes the coating of a balloon with microcapsules containing a drug.

Appropriate stents, balloons, and catheters for use in the invention can be selected by those of skill in this art. For example, such devices are available from companies such as Guidant Corporation (Indianapolis, Ind.), Boston Scientific Corporation (Natick, Mass.), and Cordis Corporation (Miami, Fla.). The stents can be made of, for example, stainless steel, tantalum, gold, titanium (e.g., a nickel titanium alloy), nitinol, and plastic. The configuration of the stent can be, for example, a coiled spring, a braided filament, a perforated tube, a slit tube, or a zigzag. Further, the configuration, diameter, and length of a stent to be used according to the invention can be selected by those of skill in the art depending upon factors such as, for example, the size and type of the blood vessel and/or the lesion to be treated.

The halichondrin analogs that are administered using the methods and devices of the invention are pharmaceutically active macrolides. In particular, Halichondrin B is a potent anticancer agent originally isolated from the marine sponge Halichondria okadai, and subsequently found in Axinella sp., Phakellia carteri, and Lissondendryx sp. Halichondrin B has demonstrated in vitro inhibition of tubulin polymerization, microtubule assembly, beta^{S}-tubulin crosslinking, GTP and vinblastine binding to tubulin, and tubulin-dependent GTP hydrolysis and has shown in vitro and in vivo anti-cancer properties. The compounds used in the present invention are analogs of Halichondrin B that fall within the following formula:

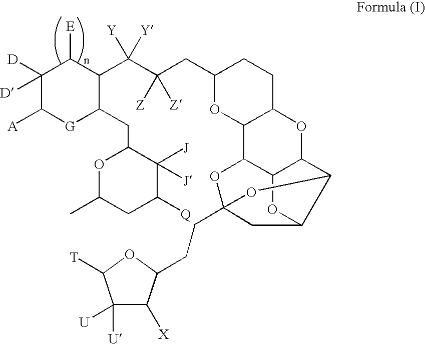

In formula (I), A is a C₁₋₆ saturated or C₂₋₆ unsaturated hydrocarbon skeleton, the skeleton being unsubstituted or having between 1 and 13 substituents, preferably between 1 and 10 substituents, e.g., at least one substituent selected from cyano, halo, azido, Q₁, and oxo. Each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁, and O(CO)NR₂R₁. The number of substituents can be, for example, between 1 and 6, 1 and 8, 2 and 5, or 1 and 4. Throughout the disclosure, numerical ranges are understood to be inclusive.

Each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl (e.g., p-fluorophenyl or p-chlorophenyl), C₆₋₁₀ hydroxyaryl, C₁₋₄ alkoxy-C₆ aryl (e.g., p-methoxyphenyl, 3,4,5-trimethoxyphenyl, p-ethoxyphenyl, or 3,5-diethoxyphenyl), C₆₋₁₀ aryl-C₁₋₆ alkyl (e.g., benzyl or phenethyl), C₁₋₆ alkyl-C₆₋₁₀ aryl, C₆₋₁₀ haloaryl -C₁₋₆ alkyl, C₁₋₆ alkyl-C₆₋₁₀ haloaryl, (C₁₋₃ alkoxy-C₆ aryl)-C₁₋₃ alkyl, C₂₋₉ heterocyclic radical, C₂₋₉ heterocyclic radical-C₁₋₆ alkyl, C₂₋₉ heteroaryl, and C₂₋₉ heteroaryl-C₁₋₆ alkyl. There may be more than one R₁, for example, if A is substituted with two different alkoxy (OR₁) groups such as butoxy and 2-aminoethyoxy.

Examples of A include 2,3-dihydroxypropyl, 2-hydroxyethyl, 3-hydroxy-4-perfluorobutyl, 2,4,5-trihydroxypentyl, 3-amino-2-hydroxypropyl, 1,2-dihydroxyethyl, 2,3-dihyroxy-4-perflurobutyl, 3-cyano-2-hydroxypropyl, 2-amino-1-hydroxy ethyl, 3-azido-2-hydroxypropyl, 3,3-difluoro-2,4-dihydroxybutyl, 2,4-dihydroxbutyl, 2-hydroxy-2(p-fluorophenyl)-ethyl, —CH₂(CO)(substituted or unsubstituted aryl), —CH₂(CO)(alkyl or substituted alkyl, such as haloalkyl or hydroxyalkyl) and 3,3-difluoro-2-hydroxypent-4-enyl.

Examples of Q₁ include —NH(CO)(CO)-heterocyclic radical or heteroaryl), —OSO₂-(aryl or substituted aryl), —O(CO)NH-(aryl or substituted aryl), aminoalkyl, hydroxyalkyl, —NH(CO)(CO)-(aryl or substituted aryl), —NH(CO)(alkyl)(heteroaryl or heterocyclic radical), O(substituted or unsubstituted alkyl)(substituted or unsubstituted aryl), and —NH(CO)(alkyl)(aryl or substituted aryl).

Each of D and D′ is independently selected from R₃ and OR₃, wherein R₃ is H, C₁₋₃ alkyl, or C₁₋₃ haloalkyl. Examples of D and D′ are methoxy, methyl, ethoxy, and ethyl. In some embodiments, one of D and D′ is H. The value for n is 1 or preferably 0, thereby forming either a six-membered or five-membered ring. This ring can be unsubstituted or substituted, e.g., where E is R₅ or OR₅, and can be a heterocyclic radical or a cycloalkyl, e.g. where G is S, CH₂, NR₆, or preferably 0. Each of J and J′ is independently H, C₁₋₆ alkoxy, or C₁₋₆ alkyl; or J and J′ taken together are ═CH₂ or —O-(straight or branched C₁₋₅ alkylene or alkylidene)-O—, such as exocyclic methylidene, isopropylidene, methylene, or ethylene. Q is C₁₋₃ alkyl, and is preferably methyl. T is ethylene or ethenylene, optionally substituted with (CO)OR₇, where R₇ is H or C₁₋₆ alkyl. Each of U and U′ is independently H, C₁₋₆ alkoxy, or C₁₋₆ alkyl; or U and U′ taken together are ═CH₂ or —O-(straight or branched C₁₋₅ alkylene or alkylidene)-O—. X is H or C₁₋₆ alkoxy. Each of Y and Y′ is independently H or C₁₋₆ alkoxy; or Y and Y′ taken together are ═O, ═CH₂, or —O-(straight or branched C₁₋₅ alkylene or alkylidene)-O—. Each of Z and Z′ is independently H or C₁₋₆ alkoxy; or Z and Z′ taken together are ═O, ═CH₂, or —O-(straight or branched C₁₋₅ alkylene or alkylidene)-O—. Also, see U.S. Pat. No. 6,214,865, the teachings of which are incorporated by reference herein in their entirety.

As a specific example, the halichondrin analog can be of the following structure:

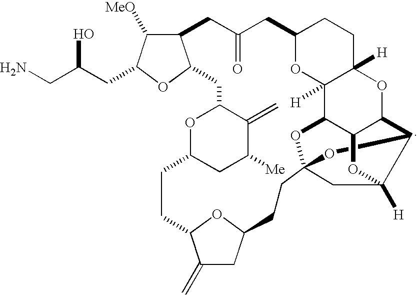

The methods and devices of the invention can be used to administer agents in addition to halichondrin analogs to patients. These agents include, for example, anticoagulants, antithrombotics, thrombolytics, antiproliferative agents, anti-inflammatory agents, anti-platelet agents, smooth muscle cell growth inhibitors, cell cycle regulating agents, antibiotics, vasodilators, and cell adhesion inhibitors. Specific examples of these and other agents that can be included in the stents of the invention, in addition to halichondrin analogs, are sirolimus/rapamycin (Wyeth-Ayerst), taxol (e.g., Paclitaxel; Angiotech, Canada), colchicine, actinomycin D, heparin and heparin fragments, streptokinase, urokilase, tissue plasminogen activator, anti-thromboxane agents, anti-B-thromboglobulin, prostaglandin E, aspirin, dipyridimol, murine monoclonal antibody 7E3, triazolopyrimidine, ciprostene, hirudin, ticlopidine, nicorandil, angiotensin converting enzyme (ACE) inhibitors, angiopeptin, cyclosporin A, terbinafine, trapidil, steroids, y-interferon, and papaverine.

All publications mentioned herein are incorporated herein by reference. Other embodiments are within the following claims.

What is claimed is:

1. A method for inhibiting the growth of cells in a blood vessel, said method comprising contacting said cells with a halichondrin analog.

2. The method of claim 1, wherein said blood vessel is a coronary artery, a vein graft, or a peripheral artery.

3. The method of claim 1, wherein said blood vessel is within a patient.

4. The method of claim 1, wherein said cells are vascular smooth muscle cells.

5. The method of claim 1, wherein said cells of said blood vessel are contacted with said halichondrin analog by use of a stent that is inserted into said blood vessel.

6. The method of claim 5, wherein said halichondrin analog is coated onto said stent.

7. The method of claim 6, wherein said halichondrin analog is present in a polymeric matrix on the surface of said stent, and said matrix facilitates release of said halichondrin analog from said matrix over time after insertion of said stent into said blood vessel.

8. The method of claim 6, wherein said stent further comprises a second therapeutic agent.

9. The method of claim 8, wherein said second therapeutic agent is selected from the group consisting of taxol, rapamycin, and heparin.

10. The method of claim 1, wherein said halichondrin analog is within the formula: 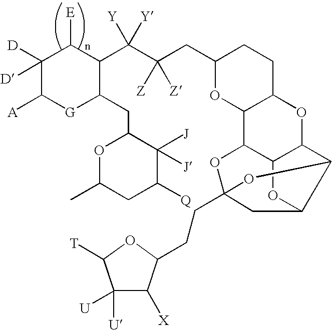
wherein A is a C₁₋₆ saturated or C₂₋₆ unsaturated hydrocarbon skeleton, said skeleton being unsubstituted or having between 1 and 10 substituents, inclusive, independently selected from cyano, halo, azido, oxo, and Q₁;
each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁, and O(CO)NR₂R₁;
each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl, C₆₋₁₀ hydroxyaryl, C₁₋₃ alkoxy-C₆ aryl, C₆₋₁₀ aryl-C₁₋₆ alkyl, C₁₋₆ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆ aryl)-C₁₋₃ alkyl, C₂₋₉ heterocyclic radical, C₂₋₉ heterocyclic radical-C₁₋₆ alkyl, C₂₋₉ hydroxyheterocyclic radical, C₂₋₉ heterocyclic radical-C₁₋₃ alkylhydroxy, C₂₋₉ heteroaryl, and C₂₋₉ heteroaryl-C₁₋₆ alkyl;
each of D and D′ is independently selected from R₃ and OR₃, wherein R₃ is H, C₁₋₃ alkyl, or C₁₋₃ haloalkyl;
n is 0 or 1;
E is R₅ or OR₅;
G is O, S, CH₂, or NR₆;
each of J and J′ is independently H. C₁₋₆ alkoxy, or C₁₋₆ alkyl; or J and J′ taken together are ═CH₂ or —O-(straight or branched C₁₋₅ alkylene)-O—;
Q is C₁₋₃ alkyl;
T is ethylene or ethenylene, optionally substituted with (CO)OR₇, where R₇ is H or C-₁₋₆ alkyl;
each of U and U′ is independently H, C₁₋₆ alkoxy, or C₁₋₆ alkyl; or U and U′ taken together are ═CH₂ or —O-(straight or branched C₁₋₅ alkylene)-O—;
X is H or C₁₋₆ alkoxy;
each of Y and Y′ is independently H or C₁₋₆ alkoxy; or Y and Y′ taken together are ═O, ═CH₂, or —O-(straight or branched C₁₋₅ alkylene)-O—; and
each of Z and Z′ is independently H or C₁₋₆ alkoxy; or Z and Z′ taken together are ═O, ═CH₂, or —O-(straight or branched C₁₋₅ alkylene)-O—;
or a pharmaceutically acceptable salt thereof.

11. The method of claim 10, wherein said halichondrin analog has the structure:

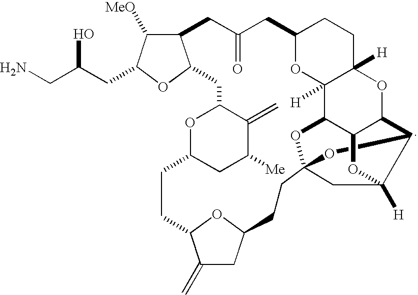

12. A stent comprising a halichondrin analog coated on its surface.

13. The stent of claim 12, wherein said halichondrin analog is present in a polymeric matrix on the surface of said stent, and said matrix facilitates release of said halichondrin analog from said matrix over time after insertion of said stent into a blood vessel.

14. The stent of claim 12, wherein said stent further comprises a second therapeutic agent.

15. The stent of claim 14, wherein said second therapeutic agent is selected from the group consisting of taxol, rapamycin, and heparin.

16. The stent of claim 12, wherein said halichondrin analog is within the formula: 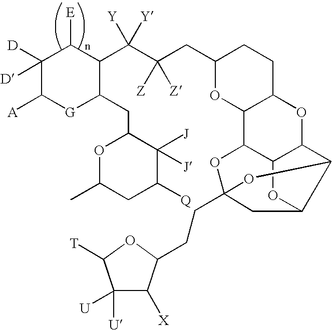
wherein A is a C₁₋₆ saturated or C₂₋₆ unsaturated hydrocarbon skeleton, said skeleton being unsubstituted or having between 1 and 10 substituents, inclusive, independently selected from cyano, halo, azido, oxo, and Q₁;
each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁, and O(CO)NR₂R₁;
each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₁₀ aryl, C₆₋₁₀ haloaryl, C₆₋₁₀ hydroxyaryl, C₁₋₃ alkoxy-C₆ aryl, C₆₋₁₀ aryl-C₁₋₆ alkyl, C₆₋₁₀ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆ aryl)-C₁₋₃ alkyl, C₂₋₉ heterocyclic radical, C₂₋₉ heterocyclic radical-C₁₋₆ alkyl, C₂₋₉ hydroxyheterocyclic radical, C₂₋₉ heterocyclic radical-C₁₋₃ alkylhydroxy, C₂₋₉ heteroaryl, and C₂₋₉ heteroaryl-C₁₋₆ alkyl;
each of D and D′ is independently selected from R₃ and OR₃, wherein R₃ is H, C₁₋₃ alkyl, or C₁₋₃ haloalkyl;
n is 0 or 1;
E is R₅ or OR₅;
G is O, S, CH₂, or NR₆;
each of J and J′ is independently H, C₁₋₆ alkoxy, or C₁₋₆ alkyl; or J and J′ taken together are ═CH₂ or —O-(straight or branched C₁₋₅ alkylene)-O—;
Q is C₁₋₃ alkyl;
T is ethylene or ethenylene, optionally substituted with (CO)OR₇, where R₇ is H or C₁₋₆ alkyl;
each of U and U′ is independently H, C₁₋₆ alkoxy, or C₁₋₆ alkyl; or U and U′ taken together are ═CH₂ or —O-(straight or branched C₁₋₅ alkylene)-O—;
X is H or C₁₋₆ alkoxy;
each of Y and Y′ is independently H or C₁₋₆ alkoxy; or Y and Y′ taken together are ═O, ═CH₂, or —O-(straight or branched C₁₋₅ alkylene)-O—; and
each of Z and Z′ is independently H or C₁₋₆ alkoxy; or Z and Z′ taken together are ═O, ═CH₂, or —O-(straight or branched C₁₋₅ alkylene)-O—;
or a pharmaceutically acceptable salt thereof.

17. The stent of claim 16, wherein said halichondrin analog has the structure:

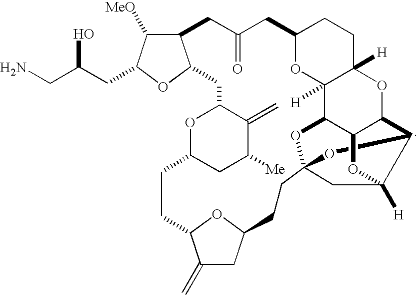

18. A method for decreasing the risk of restenosis in a coronary artery or a bypass graft of a patient, said method comprising inserting into said artery or bypass graft a stent that is coated with a halichondrin analog.
